(19) United States
(12) Patent Application Publication
(10) Pub. No.: US 2021/0292740 A1
(43) Pub. Date: Sep. 23, 2021

(54) METHODS FOR VIRAL INACTIVATION OF HUMAN PLATELET LYSATE

(71) Applicant: Arteriocyte, Inc., Hopkinton, MA (US)

(72) Inventors: Meghan E. Samberg, Upton, MA (US); Stephen E. Fischer, Shorewood, WI (US); Yiwei Ma, Belmont, CA (US); Patrick B. Patterson, Pittsfield, MA (US); Samantha A. Reilly, Chicago, IL (US); Joyce A. Howard, Shrewsbury, MA (US); Ahalya Selvaraj, Akron, OH (US); Caitlin D. Peterson, Willowick, OH (US); Donald J. Brown, Hopkinton, MA (US)

(21) Appl. No.: 17/204,072

(22) Filed: Mar. 17, 2021

Related U.S. Application Data
(60) Provisional application No. 62/990,738, filed on Mar. 17, 2020.

(51) Int. Cl. C12N 13/00; C12N 5/0775; A61K 35/19; A61P 17/02; A61P 27/02

(52) U.S. Cl. CPC C12N 13/00; C12N 5/0662; C12N 2500/84; A61P 17/02; A61P 27/02; A61K 35/19

(57) ABSTRACT

Disclosed herein are methods of inactivating viruses that may be present in human platelet lysate (hPL) by exposure to ionizing radiation. Surprisingly, the hPL retains an acceptable amount of bioactivity without requiring freeze-drying or the addition of a stabilizer. The hPL can be used as a supplement for in vitro culture of various cell types, especially human mesenchymal stromal cells (hMSCs) and for various therapeutic applications.

### CROSS-REFERENCE TO RELATED APPLICATIONS

This application claims priority to U.S. Provisional Patent Application Ser. No. 62/990,738, filed on Mar. 17, 2020, which is incorporated by reference in its entirety.

### BACKGROUND

The present disclosure relates to methods for virally inactivating fibrinogen-reduced human platelet lysate from a large donor pool. This disclosure also relates to the virally inactivated human platelet lysate obtained using such methods, cell culture methods, and therapeutic applications using such virally inactivated human platelet lysate.

Cell-based therapies are being developed for a range of diseases, including immune disorders, cancer, heart disease, and diabetes. Xenogeneic components are frequently required for isolation of desired cell populations, ex vivo culture and expansion of cells, or cryopreservation of the final cellular products. Fetal bovine serum (FBS), in particular, is widely used in cell therapy manufacturing as a supplement to basal cell culture media. FBS provides a rich source of proteins, including attachment factors, hormones, and growth factors, that support cell adhesion, growth, and proliferation.

However, FBS carries the risk of transmitting adventitious agents like bovine spongiform encephalopathy (BSE), bovine viral diarrhea virus (BVDV), and as yet unknown pathogens. FBS can also produce an immune response in some subjects when bovine components are not completely removed during cell processing or when bovine-derived antigens (e.g., N-glycolylneuraminic acid) become internalized and expressed on the surface of FBS-cultured cells.

It would be desirable to provide supplements for cell manufacturing that are safe, effective, free of xenogeneic components, and also free of pathogen such as viruses.

### BRIEF DESCRIPTION

Disclosed in the present disclosure are methods for treating human platelet lysate (hPL) to inactivate viruses. Exposure to gamma irradiation is a common method for inactivating viruses in a range of biomedical products, but, in the case of proteins, can cause denaturation or other chemical changes that alter protein function. It was surprisingly discovered that a large donor pool, fibrinogen-reduced platelet lysate having been subjected, in a frozen state, to a high dose of gamma irradiation retains satisfactory biological activity without requiring freeze-drying or addition of a stabilizer.

In a first aspect, the present disclosure relates to methods for virally inactivating hPL in the frozen state using exposure to gamma irradiation at a dose that substantially destroys common viruses yet preserves a significant bioactivity of the hPL.

In a second aspect, the present disclosure relates to the virally inactivated hPL obtained using such methods.

In a third aspect, the present disclosure relates to methods for culturing cells, particularly human mesenchymal stromal cells (hMSCs), comprising contacting said cells with a nutrient composition comprising a base medium and a virally inactivated hPL prepared as described herein.

In a fourth aspect, the present disclosure relates to methods for treating or preventing diseases or injuries, particularly skin, ocular, and sensory systems (e.g., vision, hearing, and smell), comprising administering to a person or animal an effective amount of the virally inactivated hPL as a pharmaceutical composition.

These and other non-limiting characteristics of the disclosure are more particularly disclosed below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following is a brief description of the drawings, which are presented for the purposes of illustrating the exemplary embodiments disclosed herein and not for the purposes of limiting the same.

FIGS. 1A-1C are images of a human patient's diabetic foot ulcer (DFU) healing with application of human platelet lysate-containing bandages. FIG. 1A is a picture at the start of treatment, FIG. 1B is after 16 days of treatment, and FIG. 1C is after 11 weeks of treatment. The human platelet lysate (hPL) was virally inactivated.

FIGS. 2A-2C are images of a human patient's deep partial thickness burn wound healing with application of human platelet lysate-containing bandages. FIG. 2A is a picture at the start of treatment, FIG. 2B is after 5 days of treatment, and FIG. 2C is after 15 days of treatment. The hPL was virally inactivated.

FIGS. 3A-3B are images of accelerated ocular wound healing in a guinea pig superficial ocular injury model when human platelet lysate is used as a treatment. FIG. 3A is a set of images at time 0 hrs, 24 hrs, 48 hrs, and 72 hrs for both a 20% hPL treatment and a BSS control. FIG. 3B is the set of images with fluorescein staining. The hPL was not virally inactivated, but shows a possible application of hPL prepared according to the present disclosure.

### DETAILED DESCRIPTION

A more complete understanding of the components, processes and apparatuses disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the terms “comprise(s),” “include(s),” “having,” “has,” “can,” “contain(s),” and variants thereof, as used herein, are intended to be open-ended transitional phrases, terms, or words that require the presence of the named components/ingredients/steps and permit the presence of other components/ingredients/steps. However, such description should be construed as also describing systems or devices or compositions or processes as “consisting of” and “consisting essentially of” the enumerated components/ingredients/steps, which allows the presence of only the named components/ingredients/steps, along with any unavoidable impurities that might result therefrom, and excludes other components/ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

A value modified by a term or terms, such as “about” and “substantially,” may not be limited to the precise value specified. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number.

The present disclosure may refer to temperatures for certain process steps. It is noted that these generally refer to the temperature at which the heat source (e.g. furnace or freezer) is set, and do not necessarily refer to the temperature which must be attained by the material being exposed to the heat/cold.

The term “blood” is used generally to refer to whole blood, which is a water-based fluid containing diverse solutes, suspended polypeptides, growth factors, and blood cells. Examples of blood cells are white blood cells, red blood cells and thrombocytes. White blood cells (leukocytes) are immunocompetent cells in the circulation of blood characterized by their central role of maintaining the humoral and innate immune systems. Red blood cells (erythrocytes) are the major oxygen carrying cells in the blood. The thrombocytes (platelets) are smaller than both white and red blood cells and mediate certain types of coagulation of blood. In some examples, the blood is mammalian blood, such as for example human blood.

The term “plasma” refers to the yellow liquid component of whole blood, in which the blood cells in whole blood would normally be suspended. Put another way, plasma is whole blood minus the blood cells (white, red, and thrombocytes). Plasma is mostly water and comprises dissolved proteins, glucose, clotting factors, mineral ions, hormones and carbon dioxide. Plasma may be prepared by spinning a tube of fresh blood containing an anti-coagulant in a centrifuge until the blood cells fall to the bottom of the tube. The plasma is then poured or drawn off from the blood cells.

The term “substrate” is used herein to refer to a material that is in the solid phase at room temperature, and that provides a surface upon which other materials may be adhered. The term “liquid” refers to a material that is in the liquid phase at room temperature and that provides a medium in which other materials may be suspended or dissolved, e.g. water.

The term “human platelet lysate” or “hPL” refers to the product obtained after disintegration of the cell membrane of human platelets and isolation of all molecules normally contained inside the platelets (growth factors, cytokines, etc).

The term “viral inactivation” or “virally inactivated” refers to a process or state in which the titer of a contaminating virus present in human platelet lysate has been reduced to a safe level using a treatment such as gamma irradiation.

The term “fibrinogen reduced hPL” refers to the hPL collected after a process in which the level of fibrinogen has been reduced at least 80% from the starting level of fibrinogen present in the hPL before being processed, as measured by enzyme linked immunosorbent assay (ELISA).

The term “large donor pool hPL” refers to hPL manufactured by pooling individual platelet units from at least 15 donors.

The term “base medium” denotes a medium intended for cell culture such as RPMI, MEM, DMEM medium, or a mixture of these media. These base media essentially comprise mineral salts, glucose, amino acids, vitamins and nitrogenous bases, and their ingredients and amounts are known in the art.

The term “subject” is used to refer to a person or animal who is treated with human platelet lysate for a particular condition for disease, and should not be construed as requiring any other particular circumstance to be present. For example, the person or animal does not need to be under the care of a doctor or to be part of an approved experiment.

The term “stabilizer” is used to refer to a compound that is known to reduce damage to a material when irradiated with ionizing radiation. Examples of such stabilizers can include antioxidants and free radical trapping agents. The term is used only to refer to compounds that stabilize with respect to radiation, and not to other kinds environmental conditions to which the material may be exposed.

Human platelet lysate (hPL) is a protein-rich supplement that is produced via freeze-thaw lysis of human platelet concentrates, and is an excellent option to replace FBS for multiple reasons. hPL consists of a broad spectrum of important growth factors and other active molecules useful for cell culture, including platelet-derived growth factor (PDGF), epithelial growth factor (EGF), vascular endothelial growth factor (VEGF), basic fibroblast growth factor (bFGF), hepatocyte growth factor (HGF), and transforming growth factor-β1 (TGF-β1). As a human-derived product, hPL does not harbor the risk of xenogeneic immune reactions or infections with bovine pathogens. hPL has been shown to promote efficient proliferation and/or migration of a range of human cell types, including human bone marrow-derived mesenchymal stromal cells (hMSCs), human adipose-derived stromal cells, human umbilical vein endothelial cells, keratinocytes, and fibroblasts.

Although the general process for preparing hPL is relatively straightforward, published manufacturing methods vary greatly in terms of the source of platelets, number of freeze-thaw cycles, number of platelet units pooled, etc., and this can lead to batch-to-batch compositional differences. For instance, in one study, donor age was shown to impact the ability of hPL to promote proliferation and differentiation of hMSCs. Thus, different hPL lots produced from small donor pools biased towards particular age groups can be expected to show performance variations, confounding efforts aimed at standardizing xenogenic-free cell culture protocols. Pooling together a large number of individual platelet units to manufacture hPL reduces batch-to-batch compositional differences and ensures the hPL performs consistently.

Additionally, most hPL recipes require the addition of heparin to prevent hPL from clotting upon contact with calcium in culture medium. Not only is commercially available heparin derived from porcine sources (nullifying efforts to remove all xenogeneic supplements), but using hPL in this form does not remove fibrinogen, which has been shown to negatively affect the immunomodulatory functions of cultured hMSCs.

By sourcing platelet units only from FDA-registered blood banks, which are required to conduct platelet collections under strict donor screening criteria and to test all platelet units for transmissible diseases, a high level of safety can be ensured for hPL. Despite using only platelet units that have undergone rigorous infectious disease screening, however, contamination of hPL with undiscovered and emerging pathogens is always possible. Pooling together a large number of individual platelet units to ensure hPL consistency further increases this risk. The potential contamination of hPL with pathogens, particularly viruses, represents the most significant safety concern for use of hPL in cell culture. Filtration, even with filters as small as 0.22 micrometers (μm), is not sufficient to remove viruses, which can have even smaller sizes.

Exposure to gamma irradiation is a common method for inactivating viruses in a range of biomedical products, but, in the case of proteins, can cause denaturation or other chemical changes that alter protein function. Surprisingly, it was observed that a large donor pool, fibrinogen-reduced platelet lysate having been subjected, in a frozen state, to a high dose of gamma irradiation retains a satisfactory biological activity without requiring freeze-drying or addition of a stabilizer.

Making of Platelet Lysate

Human platelet lysate (hPL) is a cell-free formulation of platelet-derived factors produced via a simple freeze-thaw lysis process. Generally, during processing of the platelets, all clotting factors and cellular membranes are removed via centrifugation and filtration, leaving behind a growth factor-rich preparation with a very low content of white blood cell antigens that could cause immune responses. hPL also contains a plethora of growth factors known to enhance cell proliferation and angiogenesis, including VEGF, PDGF, bFGF, TGF-β, and EGF. hPL also provides a supraphysiological dose of platelet factors.

Generally, human platelet lysate (hPL) is obtained from human platelets obtained from apheresis method or some other form of blood donation. The platelets are either fresh (i.e. suitable for transfusion) or expired (i.e. stored for 5 days or more after the preparation thereof and no longer suitable for transfusion).

The platelets are usually provided as a suspension in a liquid medium comprising plasma. Typically, both the red blood cells and white blood cells have been removed, and are either not present in such a suspension or are present at very low levels. A calcium chelator, such as acid citrate dextrose (ACD), is typically added to the platelet suspension during collection from a donor to prevent coagulation, which involves the conversion of free fibrinogen to fibrin.

First, the platelet suspension is subjected to at least one cycle of freezing the platelet suspension, then thawing the frozen suspension so as to obtain a lysed platelet composition. The freezing/thawing cycle induces the destruction of the platelets with the release of the contents thereof, and particularly of the endogenous factors thereof. The platelet suspension can be frozen at a temperature ranging from about −10° C. to about −80° C. The frozen platelet suspension can be thawed at a temperature ranging from about 4° C. to about 37° C.

Second, fibrinogen present in the lysed platelet suspension is precipitated for the purposes of its removal. In this regard, the blood plasma in which the platelets are suspended contains soluble fibrinogen. The normal concentration of soluble fibrinogen in blood plasma is about 150 to about 400 milligrams per deciliter (mg/dl). Normally, the soluble fibrinogen present in plasma would automatically be converted to insoluble fibrin upon removal from the body (such as from a platelet donation) in an enzyme-driven process known as coagulation. However, for coagulation to occur, calcium ions are needed. As previously mentioned, a calcium chelator is typically added to the platelet suspension to prevent coagulation. In this step, a calcium salt, such as calcium chloride (CaCl₂)) or calcium gluconate (C₁₂H₂₂CaO₁₄), is added to overcome the capacity of the calcium chelator and cause coagulation to occur.

Notably, it has been discovered that addition of the calcium salt alone results in the formation of a solid mass of fibrin, which can be subsequently removed, resulting in a high yield of platelet lysate liquid fraction that is substantially depleted of fibrinogen. Others have also added heparin during the fibrinogen depletion step to help control the coagulation procedure. Heparin is derived from animal sources (typically porcine), so the elimination of heparin from the fibrinogen depletion step enables the final product produced to be completely xenogeneic-free. In other words, heparin is not added in the processes of the present disclosure.

Next, the lysed platelet suspension containing precipitated fibrin is separated into two parts, a clear platelet lysate liquid fraction and a solid fraction containing the fibrin mass as well as cellular debris resulting from freeze/thaw destruction of the platelets. This separation is performed by centrifuging the lysed platelet suspension so as to obtain the clear platelet lysate liquid fraction (as supernatant) and the solid fraction (as sediment) containing the fibrin mass and cellular debris. The clear platelet lysate liquid fraction is then isolated from the solid fraction.

The clear platelet lysate liquid fraction then undergoes sterile filtration using a series of filters, where the final sterile filtration typically uses a filter with a nominal pore size of 0.22 micrometers (μm) or less. The resulting product can be considered fibrinogen reduced human platelet lysate (hPL). The fibrinogen concentration in the fibrinogen reduced hPL may be reduced by at least 80% compared to the fibrinogen concentration in the lysed platelet suspension. The fibrinogen concentration can be accurately measured using ELISA, a method well known in the art for determining protein concentrations via the use of monoclonal antibodies. In more specific embodiments, the fibrinogen concentration in the resulting clear platelet lysate liquid fraction may be reduced by at least 82%, or at least 84%, or at least 86%, or at least 88%, or at least 90%, or at least 92%, or at least 94%, or at least 96%, or at least 98%, or at least 99% compared to the fibrinogen concentration in the lysed platelet suspension.

As previously mentioned, hPL can be pooled from a large number of donors (i.e. at least 15 donors). The pooling can occur before or after any one of the processing steps described above.

Viral Inactivation of Human Platelet Lysate

The hPL (provided in the liquid state) is then virally inactivated by freezing and exposure to ionizing radiation in the frozen state.

First, the liquid hPL is frozen to obtain a frozen platelet lysate. The freezing of the liquid hPL is carried out at a temperature from about −10° C. to about −80° C.

It is noted that freezing differs from freeze-drying. In freezing, the liquid hPL is simply exposed to a low enough temperature to elicit a phase change from the liquid to the solid state. In contrast, freeze-drying requires the additional step of removing water from the frozen product utilizing a vacuum system, a process known as sublimation. Freeze-drying requires multiple additional procedures and relatively significant treatment times which thereby increase the costs associated with the use of such processes.

Next, the frozen platelet lysate is irradiated with ionizing radiation, which has been found to inactivate viruses. In particular embodiments, the ionizing radiation is gamma radiation. Gamma radiation is a form of highly energetic electromagnetic radiation that has a very short wavelength of less than one-tenth of one nanometer. In particular embodiments, the irradiation is carried out at an absorbed dose in the range of from about 10 kilograys (kGy) to about 60 kGy. The volume of hPL does not matter as long as it can be confirmed the entire volume has received the prescribed dose of radiation. In more particular embodiments, the absorbed dose is in the range of about 15 kGy to about 40 kGy.

The irradiation with ionizing radiation of the frozen platelet lysate is carried out while the frozen platelet lysate is maintained at a low temperature. For example, the frozen platelet lysate can be placed in dry ice during the irradiation.

It should be noted that the irradiation with ionizing radiation is in particular carried out with no exogenous stabilizer. A stabilizer is a compound that is known to reduce damage to the material to be irradiated with ionizing radiation. Examples of known stabilizers include antioxidants (ascorbic acid, tocopherol); free radical trapping agents; certain polysaccharides such as cellulose or chitosan; and certain proteins such as gelatin. Addition of such stabilizers represents an additional processing step and material which is advantageous to be done away with.

In some embodiments, the irradiation step is carried out on the frozen platelet lysate in the final packaging thereof. The final packaging is, for example, made of a material resistant to irradiation with ionizing radiation. If desired, after irradiation the frozen platelet lysate can be permitted to thaw into a liquid state.

In certain embodiments, it is contemplated that the virally inactivated hPL is then depleted of water by a process such as lyophilization (i.e. freeze-drying) to provided dehydrated products that may be combined with pharmaceutically acceptable carrier materials or excipients and used in therapeutic applications provided herein.

The resulting virally inactivated platelet lysate that is obtained after the exposure to ionizing radiation also preserves at least 80% of the bioactivity of the human platelet lysate prior to the irradiation thereof. The bioactivity is measured using a validated, cell-based functional assay whereby the growth rate of human mesenchymal stromal cells (hMSCs) in base medium supplemented with platelet lysate is quantified. In more specific embodiments, at least 82%, at least 84%, at least 86%, at least 88%, at least 90%, at least 92%, at least 94%, or at least 96% of the original bioactivity is preserved.

The growth factors PDGF, HGF, IGF, bFGF, NGF, BDNF, TGF-beta1, EGF, and VEGF are the main growth factors present in human platelet lysate (hPL). Maintaining their concentration is an indicator of maintenance of the biological activity of the virally inactivated hPL, which is critical to the use of the hPL for culturing cells and for therapeutic applications.

Under the irradiation conditions, particularly in respect of relatively high dose enabling the destruction of pathogens and of very low temperature, it is observed that surprisingly, the concentration of most of the growth factors contained in the hPL remains substantially equivalent.

Even after the irradiation with ionizing radiation, the virally inactivated hPL preserves at least 80% of the concentration of endogenous growth factors such as VEGF, PDGF-BB, HGF, IGF, bFGF, NGF, BDNF, TGF-beta1, and/or EGF. In more specific embodiments, at least 82%, at least 84%, at least 86%, at least 88%, at least 90%, at least 92%, at least 94%, or at least 96% of the original concentration is preserved.

In particular embodiments, the virally inactivated hPL has a concentration of vascular endothelial growth factor (VEGF) of from about 40 to about 4000 picograms per milliliter (pg/mL), including from about 200 to about 2000 pg/mL or from about 200 to about 900 pg/mL, or from about 500 pg/mL to about 700 pg/mL.

In particular embodiments, the virally inactivated hPL has a concentration of platelet-derived growth factor-BB (PDGF-BB) from about 500 to about 50,000 picograms per milliliter (pg/mL), including from about 2,500 to about 25,000 pg/mL or from about 8,000 to about 10,000 pg/m L.

In particular embodiments, the virally inactivated hPL has a concentration of hepatocyte growth factor (HGF) from about 30 to about 3,000 picograms per milliliter (pg/mL), including from about 150 to about 1,500 pg/m L or from about 400 pg/m L to about 500 pg/m L.

In particular embodiments, the virally inactivated hPL has a concentration of insulin-like growth factor-1 (IGF-1) from about 5 to about 500 nanograms per milliliter (ng/mL), including from about 25 to about 250 ng/mL or from about 80 ng/mL to about 100 ng/m L.

In particular embodiments, the virally inactivated hPL has a concentration of basic fibroblast growth factor (bFGF) from about 8 to about 800 picograms per milliliter (pg/mL), including from about 40 to about 400 pg/mL or from about 100 pg/mL to about 150 pg/m L.

In particular embodiments, the virally inactivated hPL has a concentration of nerve growth factor (NGF) from about 10 to about 1,000 picograms per milliliter (pg/mL), including from about 50 to about 500 pg/mL or from about 150 to about 200 pg/mL.

In particular embodiments, the virally inactivated hPL has a concentration of brain-derived neurotrophic factor (BDNF) from about 5 to about 500 nanograms per milliliter (ng/mL), including from about 25 to about 250 ng/mL or from about 80 to about 100 ng/m L.

In particular embodiments, the virally inactivated hPL has a concentration of transforming growth factor beta 1 (TGF-β1) from about 8 to about 800 nanograms per milliliter (ng/mL), including from about 40 to about 400 ng/mL or from about 120 to about 150 ng/m L.

In particular embodiments, the virally inactivated hPL has a concentration of EGF from about 150 to about 15,000 picograms per milliliter (pg/mL), including from about 750 to about 7,500 pg/mL or from about 2,400 to about 3,400 pg/m L.

Any combination of the concentration of these endogenous growth factors (whether in m/v or in % terms) is contemplated. In one specific combination, the virally inactivated hPL has a VEGF concentration of about 500 pg/mL to about 700 pg/mL; a bFGF concentration of about 100 pg/mL to about 150 pg/mL; an EGF concentration of about 2,400 pg/mL to about 3,400 pg/mL; and a PDGF-BB concentration of about 8,000 pg/m L to about 10,000 pg/mL;

In another specific combination, the virally inactivated hPL has a VEGF concentration of at least 90%, a bFGF concentration of at least 82%, an EGF concentration of at least 96%, and a PDGF-BB concentration of at least 92% of the original concentration of these growth factors prior to irradiation.

The degree of viral reduction in the virally inactivated hPL may be described in terms of the viral log₁₀ reduction factor (LRF) of model viruses before and after irradiation, as described further herein. In this regard, relevant viruses of concern for human blood-based products include hepatitis A virus (HAV), hepatitis B virus (HBV), hepatitis C virus (HCV), human immunodeficiency virus (HIV), and parvovirus B19 (B19). Other viruses are conventionally used as models or proxies for these viruses due to ease/difficulty of culture.

The LRF for pseudorabies virus (PRV) or duck hepatitis B virus (DHBV) may be 2 or more, or 3 or more, or 4 or more, or 5 or more. An upper limit for this LRF may be 10. Other enveloped DNA viruses might also serve as models to indicate the degree of inactivation of hepatitis B virus (HBV).

The LRF for encephalomyocarditis virus (EMCV) or hepatitis A virus (HAV) may be 2 or more, or 3 or more, or 4 or more. An upper limit for this LRF may be 10. Other non-enveloped RNA viruses might also serve as models to indicate the degree of inactivation of hepatitis A virus.

The LRF for bovine viral diarrhea virus (BVDV), West Nile virus (WNV), tick-born encephalitis virus (TBEV), yellow fever virus (YFV), sindbis virus (SINV), or Semliki Forest virus (SFV) may be 2 or more, or 3 or more, or 4 or more, or 5 or more. An upper limit for this LRF may be 10. Other enveloped RNA viruses might also serve as models to indicate the degree of inactivation of hepatitis C virus (HCV).

The LRF for human immunodeficiency virus (HIV) may be 2 or more, or 2.5 or more. An upper limit for this LRF may be 5.

The LRF for porcine parvovirus (PPV), canine parvovirus (CPV), minute virus of mice (MVM), or bovine parvovirus (BPV), may be 2 or more. An upper limit for the LRF may be 5. Other small, non-enveloped DNA viruses might also serve as models to indicate the degree of inactivation of parvovirus B19 (B19).

Any combination of these LRFs for the listed viruses is contemplated. Very generally, the LRF for HBV is 2 or more; the LRF for HAV is 2 or more; the LRF for HCV is 2 or more; the LRF for HIV is 2 or more; and the LRF for B19 is 2 or more.

In one specific combination, the LRF for PRV is 2 or more; the LRF for EMCV is 2 or more; the LRF for BVDV is 2 or more; the LRF for HIV is 2 or more; and the LRF for PPV is 2 or more. In another combination, the LRF for PRV is 5 or more; the LRF for EMCV is 4 or more; the LRF for BVDV is 5 or more; the LRF for HIV is 2 or more; and the LRF for PPV is 2 or more.

The virally inactivated human platelet lysate (hPL) retains its efficacy in promoting proliferation of multiple cell types. Thus, the hPL can be used to culture cells, such as human mesenchymal stromal cells (hMSCs) obtained from bone marrow or from umbilical cord blood or from adipose tissue.

In some embodiments, the virally inactivated hPL is mixed with a base medium to form a nutrient composition. Cells are then contacted with the nutrient composition.

The base medium may be any conventional cell culture medium, such as RPMI 1640, MEM, DMEM, or mixtures thereof. These base media essentially comprise mineral salts, glucose, amino acids, vitamins, and nitrogenous bases.

The base medium may comprise from about 75% to about 98% by volume of the nutrient composition. The virally inactivated hPL may comprise from about 2% to about 25% by volume of the nutrient composition.

In this regard, cells typically require two things to be cultured: (1) a substrate that provides a structural support for the cell; and (2) a cell culture medium to provide nutrition to the cell. For example, a petri dish or other container is usually used as the substrate for in vitro applications. The nutrient composition is intended to be used as a cell culture medium.

In particular embodiments, the growth rate of hMSCs in the nutrient composition comprising a base medium and virally inactivated hPL is at least 80% of the growth rate of hMSCs in a nutrient composition comprising the same base medium and hPL that has not been exposed to ionizing radiation. The growth rate is measured by quantifying the total hMSC population at any point during a culture and comparing this to the population at the start of the culture. The hMSC population may be quantified by monitoring such parameters as DNA content, DNA synthesis, metabolic activity, protease activity, tracking dye intensity, and any number of other methods known in the field. In more specific embodiments, the growth rate of hMSCs in the nutrient composition comprising the virally inactivated hPL is at least 82%, at least 84%, at least 86%, at least 88%, at least 90%, at least 92%, at least 94%, or at least 96% of the growth rate of hMSCs in the nutrient composition comprising the hPL that has not been exposed to ionizing radiation.

In other embodiments, the virally inactivated hPL may be combined with pharmaceutically acceptable carrier materials or excipients for use in therapeutic applications. The virally inactivated hPL may simply be combined in the liquid state with such materials or, as mentioned previously, may be first dehydrated prior to combining with such materials. Particular carrier materials may include biomaterial scaffolds designed to deliver the hPL in a controlled manner to the target location, provide structure to support the ingrowth of regenerating tissue, or a number of other important functions to support the particular therapeutic application. The scaffolds may be fabricated from natural materials, such as proteins or polysaccharides, or from non-natural materials, such as synthetic polymers. The scaffolds may take a number of different forms, including fibrous, foam, hydrogel, microsphere, or composites thereof.

Particular excipients may include buffering agents, surfactants, preservative agents, bulking agents, polymers, and stabilizers, which are useful with these molecular antagonists. Buffering agents are used to control pH. Surfactants are used to stabilize proteins, inhibit protein aggregation, inhibit protein adsorption to surfaces, and assist in protein refolding. Exemplary surfactants include Tween 80, Tween 20, Brij 35, Triton X-10, Pluronic F127, and sodium dodecyl sulfate. Preservatives are used to prevent microbial growth. Examples of preservatives may include benzyl alcohol, m-cresol, and phenol. Bulking agents are used during lyophilization to add bulk. Hydrophilic polymers such as dextran, hydroxyl ethyl starch, polyethylene glycols, and gelatin can be used to stabilize proteins. Polymers with nonpolar moieties such as polyethylene glycol can also be used as surfactants. Protein stabilizers can include polyols, sugars, amino acids, amines, and salts. Suitable sugars include sucrose and trehalose. Amino acids include histidine, arginine, glycine, methionine, proline, lysine, glutamic acid, and mixtures thereof. Proteins like human serum albumin can also competitively adsorb to surfaces and reduce aggregation of the protein-like molecular antagonist. It should be noted that particular molecules can serve multiple purposes. For example, histidine can act as a buffering agent and an antioxidant. Glycine can be used as a buffering agent and as a bulking agent. The dehydrated hPL (with excipients) may then be reconstituted with, for example, suitable diluents such as normal saline, sterile water, glacial acetic acid, sodium acetate, combinations thereof and the like.

The virally inactivated hPL can be used for several therapeutic applications. In particular, an effective amount of the virally inactivated hPL can be administered as a pharmaceutical composition for treating or preventing diseases or injuries, particularly those to the dermal, ocular, or sensory systems (e.g., vision, hearing, and smell). The term “treat” is used to refer to a reduction in progression of the disease/injury, a regression in the disease/injury, and/or a prophylactic usage to reduce the probability of presentation of the disease/injury. Such dosages can be determined.

Dermal wounds, particularly those considered chronic wounds, cause pain, hinder functional recovery from injury, impair quality of life, and lead to serious and often fatal infections. It has been reported that pooled, allogeneic hPL can induce viability, proliferation, migration, and angiogenic activity of human cells involved in the different phases of dermal wound healing. Thus, an effective amount of the virally inactivated hPL may be applied to the damaged skin of a subject for therapeutic purposes.

Dry eye is a major complication associated with chronic graft-versus-host disease (GvHD) after hematopoietic stem cell transplantation. Corneal ulcers caused by physical or chemical trauma or disease can cause significant pain and vision impairment and can be difficult to treat. It has been reported that hPL is useful for the treatment of corneal ulcers caused by a range of conditions. hPL may potentially be useful for the treatment of other eye diseases too. Thus, an effective amount of the virally inactivated hPL may be applied to the eye of a subject for therapeutic purposes.

Hearing impairment and tinnitus (i.e., ringing in the ears) can be caused by disease or trauma to the auditory nerves in the inner ear. Likewise, glaucoma and other vision impairments can be caused by disease or trauma to the retina or optical nerve and anosmia (i.e., loss of smell) can be caused by trauma to olfactory receptor neurons. It has been found that hPL contains a range of important neurotrophic factors and is capable of promoting rapid neurite outgrowth. Thus, an effective amount of the virally inactivated hPL may be applied to the inner ear, eye, or nasal passage of a subject for therapeutic purposes to treat hearing loss, tinnitus, vision impairment, anosmia, and other nerve-related injuries.

The following examples are provided to illustrate the processes and compositions of the present disclosure. The examples are merely illustrative and are not intended to limit the disclosure to the materials, conditions, or process parameters set forth therein.

### Examples

Preparation of a Fibrinogen-Reduced, Large Donor Pool Human Platelet Lysate

A batch of fibrinogen-reduced, large donor pool human platelet lysate was prepared by first acquiring ˜200 expired apheresis platelet units from FDA-registered blood banks in the United States. These units were stored frozen until use. To initiate production, all units were thawed in a 4° C. refrigerator, then CaCl₂) was added to a final concentration of 20 mM to precipitate the coagulation components present in donor plasma. The CaCl₂)-spiked units were placed back in the 4° C. refrigerator for a period of 72 hours to complete the precipitation process, then re-frozen. After thawing the units a second time, a centrifugation step was carried out to pellet the coagulation solids as well as any cellular debris resulting from the freeze-thaw destruction of platelet membranes. Individual lysed platelet units were pooled into a large, sterile biocontainer, then pumped through a sterilizing filter assembly into a second sterile biocontainer. The final sterilizing filter had an absolute pore size of 0.2 μm. From the biocontainer, filter-sterilized product was distributed into final containers, labeled, and frozen.

Irradiation of Human Platelet Lysates

The frozen human platelet lysate was shipped to a contract gamma irradiation facility using insulated shippers and a sufficient quantity of dry ice to maintain it in a frozen state during shipping and irradiation. The product shipping configuration was fixed in terms of the type of packaging material used, the number and arrangement of product containers within the shipping box, and the mass of dry ice used. The shipping containers were exposed to the gamma source in a manner that resulted in a minimum dose of 25 kGy of gamma irradiation reaching all areas inside the shipping container. Prior to sending product to the contract irradiator, a dose mapping study was performed using surrogate materials to establish the ratio of the internal delivered dose using this shipping configuration to a reference location on the outside of the irradiation container. This is done because dosimeters placed with product inside a shipper containing dry ice cannot accurately measure delivered dose.

Viral Inactivation

To demonstrate that a dose of 25 kGy of gamma irradiation could inactivate viruses potentially contaminating human platelet lysate, an array of RNA and DNA model viruses, including pseudorabies virus (PRV), encephalomyocarditis virus (EMCV), bovine viral diarrhea virus (BVDV), human immunodeficiency virus (HIV), and porcine parvovirus (PPV) were spiked into liquid human platelet lysate. The virus-spiked human platelet lysate was then frozen and shipped on dry ice to a facility for exposure to gamma irradiation at a minimum dose of 25 kGy. The actual dose received by the product ranged from 27.0 to 28.8 kGy. The irradiated product was thawed and assayed for viral titer using standard virology protocols for plaque assays and tissue culture infectious dose (TCID₅₀). Samples from the same batch of human platelet lysate were also spiked with viruses, but not exposed to gamma irradiation. Viral log₁₀ reduction factors (LRFs) were calculated as: LRF=log₁₀ ([volume*titer for no gamma treatment]/[volume*titer for gamma treatment]). The results were as follows:

TABLE 1
Log reduction factors (LRFs) for three model viruses spiked into
human platelet lysate after exposure to 25 kGy gamma irradiation.

| Virus | Family | Genome | Envelope | Size (nm) | LRF |
|---|---|---|---|---|---|
| PRV | Herpes | DNA | Yes | 120-200 | >=5.42 |
| EMCV | Picorna | RNA | No | 25-30 | 4.94 |
| BVDV | Flavi | RNA | Yes | 50-70 | >=5.13 |
| HIV | Lenti | RNA | Yes | 80-100 | 2.90 |
| PPV | Parvo | DNA | No | 18-24 | 2.20 |

The results demonstrated that gamma irradiation of hPL at a dose of at least 25 kGy was capable of inactivating a range of model DNA and RNA viruses.

Assay of Growth Factors

Three batches of human platelet lysate that were previously exposed to gamma irradiation at a minimum dose of 25 kGy were thawed and analyzed for the following growth factors using a quantitative sandwich-style ELISA technique: VEGF, bFGF, EGF, and PDGF-BB. Samples of the same three batches prior to irradiation were also analyzed. ELISA was performed using commercially available Quantikine® ELISA kits from R&D Systems (Minneapolis, Minn.) following the manufacturer's recommended protocol. Briefly, monoclonal antibodies specific for each growth factor were pre-coated onto microplates. After pipetting standards and samples into the wells, unbound substances were washed away via multiple rinses with buffer. A horseradish peroxidase enzyme-linked antibody also specific to the growth factor was then added to the wells. Following a wash to remove any unbound antibody-enzyme reagent, a substrate solution consisting of tetramethylbenzidine was added to the wells and color developed in proportion to the amount of growth factor present. The color development was stopped using sulfuric acid and the intensity of the color was measured with a microplate reader set in absorbance detection mode. Growth factor concentrations were determined by comparing absorbance readings for platelet lysate samples against a standard curve. The results were as follows:

TABLE 2
Growth factor concentrations in gamma irradiated
and non-irradiated human platelet lysate

| | | | | | % remaining |
|---|---|---|---|---|---|
| | Gamma Irradiated | | Non-irradiated | | after |
| | hPL | | hPL | | Irradiation |
| VEGF | 599.4 ± 42.4 | pg/mL | 643.1 ± 26.4 | pg/mL | 93.2 |
| bFGF | 138.5 ± 5.0 | pg/mL | 164.0 ± 9.4 | pg/mL | 84.5 |
| EGF | 3001.4 ± 208.9 | pg/mL | 2798.3 ± 162.2 | pg/mL | 107.3 |
| PDGF-BB | 8638.4 ± 485.6 | pg/mL | 8704.7 ± 743.7 | pg/mL | 96.1 |

The data demonstrated that the levels of several key growth factors in hPL did not significantly decrease after exposure to gamma irradiation.

Proliferation of hMSCs

Three batches of human platelet lysate that were previously exposed to gamma irradiation at a minimum dose of 25 kGy were thawed and evaluated for their capacity to promote proliferation of hMSCs. Samples of the same three batches prior to irradiation were also evaluated. Both were compared to a commercially obtained fetal bovine serum (FBS). hMSCs were derived from human bone marrow using the standard plastic adhesion method. In preparation for the proliferation assay, an aliquot of cryopreserved hMSCs was rapidly thawed at 37° C. and suspended in a complete medium containing alpha-MEM supplemented with L-glutamine, antibiotic/antimycotic solution, and 10% by volume of either the irradiated human platelet lysate, the non-irradiated human platelet lysate, or FBS. hMSCs were seeded into 24-well plates at an initial density of 4000 cells/cm² and cultured for a period of 4 days, with a complete medium change at day 2. At the end of the culture period, the number of cells present was determined using flow cytometry. Population growth rate was estimated using the following formula: GR=[Ln(Nₜ/N₀)]/t, where GR is the approximate growth rate, Nₜ is the number of cells at the end of the culture, N₀ is the number of cells at the start of culture, and t is the total time in culture (4 days). The results were as listed in Table 3. The data demonstrated that the bioactivity of hPL was not significantly decreased after exposure to gamma irradiation.

TABLE 3
Approximate growth rate for hMSCs cultured
with different supplements.

| | Non-Irradiated | Gamma-Irradiated |
|---|---|---|
| Human Platelet Lysate | 0.018 ± 0.001 hr⁻¹ | 0.016 ± 0.001 hr⁻¹ |
| FBS | 0.010 ± 0.001 hr⁻¹ | — |

Fibrinogen Concentrations

Additional experiments were done to compare the fibrinogen concentration between the lysed platelet suspension and the fibrinogen-reduced hPL. For the lysed platelet suspension, a total of six expired platelet units were used for the testing. These units were thawed and samples were taken prior to the addition of CaCl₂). The samples were centrifuged to remove platelet debris and the supernatant was obtained. For the fibrinogen-reduced hPL, a total of five large batches of product prepared according to the methods described above were used for testing and samples were taken after the sterile filtration step. The fibrinogen concentration in all samples was evaluated using a human fibrinogen ELISA kit obtained from Abcam (Cambridge, Mass.). The results were as listed in Table 4. The data demonstrated that the fibrinogen concentration was reduced, on average, more than 99%.

TABLE 4
Reduction of fibrinogen concentration

| | Lysed Platelet Suspension | After Sterile Filtration |
|---|---|---|
| Fibrinogen | 681.3 ± 273.0 μg/mL | 1.5 ± 0.8 μg/mL |

Treatment of Skin Wounds

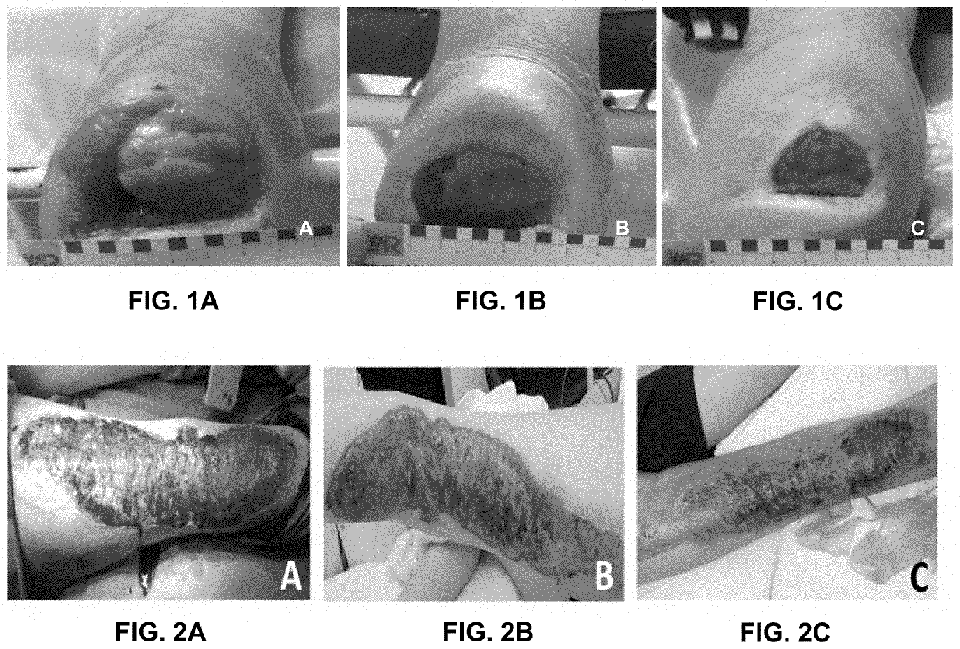

A small human study was performed to demonstrate initial safety and feasibility by applying a human platelet lysate-containing bandage to recalcitrant diabetic foot ulcers (DFUs) of a 69-year old male patient. Prior to treatment, the DFUs had persisted for greater than four months. The bandage was made by mixing human platelet lysate with bovine collagen, pouring the mixture into a 4-inch by 4-inch mold, and lyophilizing the mixture to form a porous matrix. The lyophilized 4-inch square bandage contained approximately 73% by weight human platelet lysate and 27% by weight bovine collagen. The bandage was packaged, then shipped to a facility for terminal sterilization via exposure to electron beam irradiation at a minimum dose of 15 kGy. After debriding the wound, the human platelet lysate-containing bandage was applied directly to the wound surface and then covered with an elastic outer dressing. Dressing changes were performed as needed during the treatment course; up to six human platelet lysate-containing bandages were applied per wound during the treatment course. All DFUs responded well to the treatment with some very deep wounds showing excellent healing trajectory. FIGS. 1A-1C show the effect of hPL treatment over 11 weeks.

A small human study was also performed to demonstrate initial safety and feasibility by applying a human platelet lysate-containing bandage to deep partial thickness burn wounds of a 26-year old female patient. After debriding the wound, the human platelet lysate-containing bandage was applied directly to the wound surface and then covered with an elastic outer dressing. Multiple bandages were necessary to completely cover large total body surface around wounds. Dressing changes were performed as needed during the treatment course. No adverse reactions were noted from the treatment and the wounds generally healed without the need for a skin graft. FIGS. 2A-2C show the effect of hPL treatment over 15 days. A total of six hPL-containing bandages were used.

Treatment of an Ocular Wound

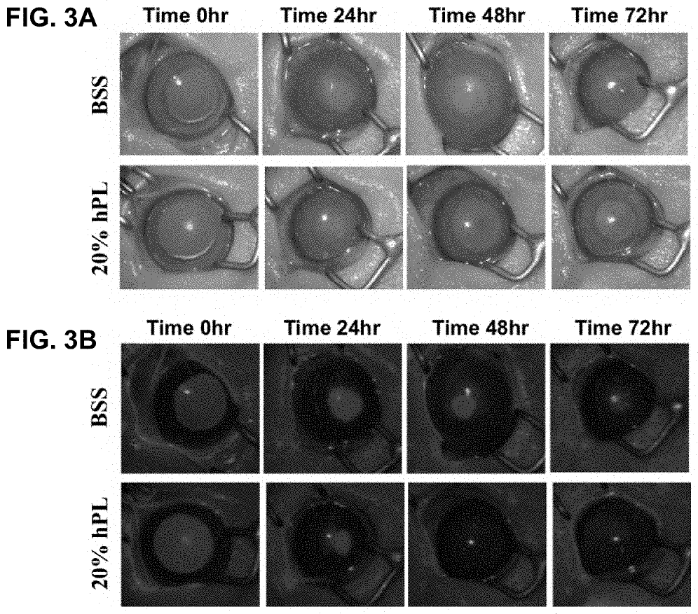

A small animal study using a guinea pig superficial ocular injury model was carried out to demonstrate the ocular wound healing potential of human platelet lysate. This hPL was not irradiated. In this model, the corneal epithelium was carefully removed using a motorized foreign body and rust ring remover brush (Algerbrush II) after demarcating the cornea with a biopsy punch. Human platelet lysate (hPL) treatments were then applied via a custom designed ocular wound chamber that prevents the animals from scratching their eyes during the study and enables treatments to maintain continuous contact with the ocular surface. FIG. 3A shows the results over 72 hours for the hPL treatments compared to controls treated with balanced salt solution (BSS). As seen in the fluorescein stained images (see FIG. 3B), a dose of 20% human platelet lysate stimulated faster epithelial closure as early as 24 hrs compared to controls treated with balanced salt solution (BSS). Complete closure with human platelet lysate was achieved by 48 hrs, whereas the control required 72 hrs.

The present disclosure has been described with reference to exemplary embodiments. Modifications and alterations will occur to others upon reading and understanding the preceding detailed description. It is intended that the present disclosure be construed as including all such modifications and alterations insofar as they come within the scope of the appended claims or the equivalents thereof.

1. A method for virally inactivating human platelet lysate (hPL), comprising:
irradiating the human platelet lysate with ionizing radiation while in a frozen state;
wherein the resulting virally inactivated hPL retains at least 80% of its original bioactivity.

2. The method of claim 1, further comprising freezing a liquid hPL to obtain the hPL in the frozen state.

3. The method of claim 1, wherein the hPL is prepared from a platelet suspension by:
subjecting the platelet suspension to at least one freezing/thawing cycle to obtain a lysed platelet composition;
depleting the lysed platelet composition of fibrinogen;
separating the lysed platelet composition into a clear platelet lysate fraction and a cellular debris fraction; and
filtering the clear platelet lysate fraction to obtain the hPL.

4. The method of claim 3, wherein the clear platelet lysate fraction is filtered with a final filter size of at least 0.22 micrometers (μm).

5. The method of claim 3, wherein the platelet suspension comprises platelets suspended in a liquid medium comprising plasma.

6. The method of claim 1, wherein the ionizing radiation is gamma radiation.

7. The method of claim 1, wherein the irradiation is carried out at an absorbed dose of from 10 kGy to 60 kGy.

8. The method of claim 1, wherein the resulting virally inactivated hPL maintains at least 80% of its original VEGF activity.

9. The method of claim 1, wherein the resulting virally inactivated hPL maintains at least 80% of its original PDGF-BB activity.

10. The method of claim 1, wherein the resulting virally inactivated hPL maintains at least 80% of its original bFGF activity.

11. The method of claim 1, wherein the resulting virally inactivated hPL maintains at least 80% of its original EGF activity.

12. The method of claim 1, wherein no stabilizer is added to the human platelet lysate prior to irradiating the human platelet lysate with ionizing radiation.

13. The virally inactivated human platelet lysate obtained by the method of claim 1.

14. A method for culturing cells, comprising contacting the cells with a nutrient composition comprising a base medium and a virally inactivated human platelet lysate (hPL).

15. A method for treating dermal wounds, dry eye, corneal ulcers, nerve-related injury, a disease causing hearing loss, tinnitus, vision loss, or anosmia in a subject, comprising:
administering an effective amount of virally inactivated human platelet lysate (hPL) to the subject.

16. The method of claim 15, wherein the virally inactivated hPL is applied to the skin, eye, inner ear, or nasal passage of the subject.
